(19) United States
(12) Patent Application Publication
(10) Pub. No.: US 2023/0000886 A1
(43) Pub. Date: Jan. 5, 2023

(54) MIXTURE OF HMOs

(71) Applicant: Glycom A/S, Hørsholm (DK)

(72) Inventors: Louise Kristine Vigsnæs, Copenhagen NV (DK); Bruce McConnell, La Tour de Peilz (CH); Dorthe Seitzberg, Espergærde (DK); Ingvild Dybdrodt Amundsen, Copenhagen S (DK)

(21) Appl. No.: 17/780,869

(22) PCT Filed: Nov. 27, 2020
(86) PCT No.: PCT/IB2020/061215

§ 371 Date: May 27, 2022

(30) Foreign Application Priority Data
Nov. 27, 2019 (DK) PA 2019 01395

(51) Int. Cl. A61K 31/702; A61P 1/12

(52) U.S. Cl. CPC A61K 31/702; A61P 1/12

(57) ABSTRACT

The invention relates to a human milk oligosaccharide (HMO) for use in, a synthetic composition comprising an HMO for use in, and a method for prophylactically reducing gastrointestinal IBS symptom severity and/or symptom occurrence in a lac-tose-intolerant, IBS patient; reintroducing a source of lactose into the diet of a lactose-intolerant, IBS patient while prophylactically reducing gastrointestinal IBS symptom severity and/or symptom occurrence in the IBS patient; and/or reducing the severity and/or occurrence of non-gastrointestinal symptoms in a lactose-intolerant, IBS patient.

### FIELD OF THE INVENTION

This invention relates to a method, compounds and composition for managing symptoms in lactose-intolerant, IBS patients.

### BACKGROUND OF THE INVENTION

Irritable bowel syndrome (IBS) is a clinically heterogeneous, functional bowel disorder with chronic symptoms such as abdominal pain, altered bowel movement, abdominal discomfort, abdominal bloating, and fatigue. Routine clinical tests on patients typically show no abnormalities, although their bowels may be more sensitive to certain stimuli, such as balloon insufflation testing. The worldwide prevalence of IBS is about 10-20% but may be higher in certain countries. The causes of IBS are unknown but disruptions of the brain-gut axis, acute gastrointestinal infections, small intestinal bacterial overgrowth, antibiotic usage and intestinal dysbiosis are thought to be important risk factors (Kim et al. Digest. Dis. Sci. 57, 3213 (2012)). Many patients appear to have a lower abundance of Bifidobacterium in their faeces than healthy controls (Pittayanon et al. Gastroenterology 157, 97 (2019)). Other risk factors are young age, prolonged fever, anxiety, and depression. Certain patients exhibit signs of chronic low-grade inflammation but there are otherwise little or no observable clinical manifestations.

Patients with IBS are more likely to report intolerance to dairy products than healthy individuals (Yang et al. Alim. Pharm. Therap. 39, 302 (2014)). Also, it has been proposed that malabsorption of easily fermented carbohydrates (such as lactose in patients with lactase deficiency) is a cause of IBS in many patients. The mechanism underlying the link between IBS and food intolerance is uncertain. However, some studies suggest a shared aetiology involving both psychological (e.g. anxiety) and gastrointestinal (GI) dysfunction (e.g. altered gut transit, visceral hypersensitivity). Lactose intolerance occurs because lactose is inefficiently digested in the small intestines. This is due to reduced expression or impaired activity of the enzyme beta-galactosidase, which is produced by the cells that line the small intestine. Beta-galactosidase breaks down lactose into two sugars; glucose and galactose, which are then transported across the cell membrane and absorbed into the bloodstream. If beta-galactosidase is not present, or not present in sufficient levels, undigested lactose passes through the small intestine into the large intestine. Here, many different bacteria such as Escherichia and Clostridium have the capacity to metabolize lactose, which they convert to acids and gasses. Hence, undigested lactose can lead to increased production of gas in the colon. In addition, undigested lactose can affect the osmotic load in the colon and change colonic motility. Collectively, these events are likely to lead to gastrointestinal symptoms such as diarrhoea, cramping, bloating and abdominal pain. In fact, 33-97% of individuals with lactose intolerance do report these symptoms after dairy consumption.

At present, there is no universally accepted therapy for the treatment of lactose intolerance. However, existing strategies for management of the conditions include avoidance of lactose-containing dairy foods (milks, soft cheeses and ice creams) and consuming lactase prior to a meal containing lactose. Although restricting dietary lactose may improve gastrointestinal complaints, long-term effects of a diet low or free of dairy products can be of concern, since dairy products provides a package of essential nutrients such as calcium, protein, riboflavin, vitamin A and vitamin D. Dietary calcium is an important part of the recommended daily allowance of vitamins and minerals, and for many people it is not possible to achieve recommended daily calcium intakes with a dairy low or free diet. The alternative is the consumption of a limited range of expensive lactose-reduced dairy product. The consumption of lactase is effective, but it must be consumed before meals and, although uncommon, there may be serious, allergy-related side effects.

While diagnosis of lactose intolerance is relatively easy using a lactose tolerance test, diagnosis of IBS is difficult. There are no reliable biomarker-based tests to diagnose IBS. Diagnosis generally involves excluding conditions that produce IBS-like symptoms and then following a procedure to categorise a patient's symptoms. Once diagnosed, patients are usually classified in accordance with the Rome IV criteria into three symptom subtypes based on stool consistency: diarrhoea predominant (IBS-D), constipation predominant (IBS-C), and mixed subtype (IBS-M) with alternating episodes of both diarrhoea and constipation. Diagnosing patients who are lactose intolerant and who suffer from IBS is even more difficult because exclusion of lactose intolerance is part of the procedure for diagnosing IBS.

Management of IBS symptoms generally focuses on attempting to relieve symptoms. Interventions take various forms such as dietary adjustments, probiotics, antibiotics, medication, and psychological interventions. However, most treatments are unsatisfactory and most patients continue to experience abdominal pain, altered bowel movements, bloating, fatigue, and other symptoms. A recent development in IBS treatment has been the low FODMAP diet. This diet requires patients to restrict the intake of FODMAP carbohydrates. These are Fermentable Oligo-, Di-, Monosaccharides And Polyols which are poorly absorbed in the small intestine, osmotically active, and fermented by intestinal bacteria producing hydrogen. Adherence to this diet has resulted in symptom improvements for some IBS patients. A low FODMAP diet should remove or reduce lactose in the diet and therefore should help lactose intolerant, IBS patient manage their symptoms. However, some of the FODMAP carbohydrates are beneficial fibres, and foods that contain them are common, highly nutritious fruits, vegetables and legumes. Further, removal of dairy products has long term nutritional consequences. Also, it is very difficult to comply with the low FODMAP diet over longer periods.

Recently, human milk oligosaccharides (HMOs) have been proposed as a possible approach to manage symptoms of IBS (WO 2016/066175). HMOs are a heterogeneous mixture of soluble glycans found in human milk. They are the third most abundant solid component after lactose and lipids in human milk and are present in concentrations of 5-25 g/I (Bode:Human milk oligosaccharides and their beneficial effects, in: Handbook of dietary and nutritional aspects of human breast milk (Zibadi et al., eds.), pp. 515-31, Wageningen Academic Publishers (2013)). HMOs are resistant to enzymatic hydrolysis in the small intestine and are thus largely undigested and unabsorbed and reach the colon intact. Most of the HMOs that reach the colon serve as substrates to shape the gut ecosystem by selectively stimulating the growth of specific bacteria. HMOs are believed to substantially modulate the infant gut microbiota and play a decisive role in the differences in the microbiota of formula-fed and breast-fed infants. These differences include the predominance of Bifidobacterium in the gut of breast-fed infants compared to a more diverse gut microbiota in formula-fed infants. This is viewed as beneficial for the infant because strains of Bifidobacterium species are believed to have a positive effect on gut health.

However, HMOs contain lactose at their reducing end; usually bounded to one or more β-N-acetyl-lactosaminyl, β-lacto-N-biosyl, α L-fucopyranosyl and/or an α-N-acetyl-neuraminyl (sialyl) moieties. Further, industrial HMOs are generally produced by fermentation where lactose is used a feed solution. Consequently, industrially produced HMOs contain certain amounts of free lactose and their use in the management of symptoms of lactose-intolerant, IBS patients is counter-intuitive.

Therefore, there remains a need for methods and compounds for the management of IBS symptoms in lactose-intolerant, IBS patients; particularly methods and compounds which permit the patients to include lactose-containing products in their diet.

### SUMMARY OF THE INVENTION

A first aspect of the invention relates to a human milk oligosaccharide (HMO) for use in:
prophylactically reducing gastrointestinal IBS symptom severity and/or symptom occurrence in a lactose-intolerant, IBS patient,
reintroducing a source of lactose into the diet of a lactose-intolerant, IBS patient while prophylactically reducing gastrointestinal IBS symptom severity and/or symptom occurrence in the IBS patient, and/or
reducing the severity and/or occurrence of non-gastrointestinal symptoms in a lactose-intolerant, IBS patient.

A second aspect of the invention relates to a synthetic composition for use in:
prophylactically reducing gastrointestinal IBS symptom severity and/or symptom occurrence in a lactose-intolerant, IBS patient,
reintroducing a source of lactose into the diet of a lactose-intolerant, IBS patient while prophylactically reducing gastrointestinal IBS symptom severity and/or symptom occurrence in the IBS patient, and/or
reducing the severity and/or occurrence of non-gastrointestinal symptoms in a lactose-intolerant, IBS patient, the composition comprising at least one human milk oligosaccharide (HMO).

Preferably the synthetic composition contains an amount of 1 g to 15 g of the HMO; more preferably 2 g to 10 g. For example, the synthetic composition may contain 3 g to 7 g of the HMO.

The synthetic composition may contain a bifidobacteria; for example, Bifidobacterium longum and/or Bifidobacterium bifidum.

A third aspect of the invention relates a pack for use in
prophylactically reducing gastrointestinal IBS symptom severity and/or symptom occurrence in a lactose-intolerant, IBS patient,
reintroducing a source of lactose into the diet of a lactose-intolerant, IBS patient while prophylactically reducing gastrointestinal IBS symptom severity and/or symptom occurrence in the IBS patient, and/or
reducing the severity and/or occurrence of non-gastrointestinal symptoms in a lactose-intolerant, IBS patient, the pack comprising at least 14 individual daily doses of an effective amount of at least one human milk oligosaccharide (HMO).

Preferably each dose in the pack contains about 1 g to 15 g of the human milk oligosaccharide, preferably 2 g to 10 g, more preferably 3 g to 7 g.

The pack preferably comprises at least about 21 daily doses; for example, about 28 daily doses.

The pack may contain a bifidobacteria; for example, Bifidobacterium longum and/or Bifidobacterium bifidum.

A fourth aspect of the invention relates to a method for prophylactically reducing gastrointestinal IBS symptom severity and/or occurrence in a lactose-intolerant, IBS patient, the method comprising administering to the patient an effective amount of at least one human milk oligosaccharide (HMO).

A fifth aspect of the invention relates to a method for reintroducing a source of lactose into the diet of a lactose-intolerant, IBS patient while prophylactically reducing gastrointestinal IBS symptom severity and/or symptom occurrence in the IBS patient, the method comprising administering to the patient an effective amount of at least one human milk oligosaccharide (HMO) prior to consumption of the source of lactose.

Preferably the human milk oligosaccharide is administered for a period of at least 1 week, more preferably at least 2 weeks, prior to consumption of the source of lactose.

Preferably the human milk oligosaccharide is additionally administered after consumption of the source of lactose.

A sixth aspect of the invention relates to a method reducing the severity and/or occurrence of non-gastrointestinal symptoms in a lactose-intolerant, IBS patient, the method comprising administering to the patient an effective amount of at least one human milk oligosaccharide (HMO).

Preferably the non-gastrointestinal symptom is one or more of trouble sleeping, headache, lack of energy, fatigue, inability to concentrate, anxiety, depression, and skin rash.

Preferably at least one human milk oligosaccharide (HMO) is administered prior to consumption of a source of lactose. More preferably, the human milk oligosaccharide is additionally administered after consumption of a source of lactose.

The amount of the HMO administered is preferably effective to increase the abundance of butyrate-producing bacteria and/or bifidobacteria in the intestine of the human. Further, the amount of the HMO administered is preferably effective to improve the intestinal barrier properties of the human, particularly in the colon.

Preferably the human is administered an amount of 1 g to 15 g per day of the HMO; more preferably 2 g to 10 g per day. For example, the human may be administered 3 g to 7 g per day. Preferably the human is administered the HMO for a period of at least 1 week; more preferably for at least 2 weeks.

The patient may be administered a higher dose during an initial phase and a lower dose during a maintenance phase. Preferably the human is administered the HMO for a period of at least 1 week; more preferably for at least 2 weeks during the initial phase. The initial phase may occur prior to consumption of a source of lactose. The human may be administered the HMO for a period of at least 4 weeks; more preferably for at least 8 weeks during the maintenance phase. The maintenance phase may occur after consumption of the source of lactose. The dose administered during an initial phase is preferably about 3 g to about 10 g per day (for example about 4 g to about 7.5 g per day) and the dose administered during a maintenance phase is preferably about 2 g to about 7.5 g per day (for example about 2 g to about 5 g per day).

In certain embodiments, the HMO can be a neutral HMO or an acidic HMO. The neutral HMO can be one or more fucosylated HMOs or one or more non-fucosylated HMOs. Preferably the HMO is selected from 2′-FL, 3-FL, DFL, LNT, LNnT, 3′-SL, 6′-SL, LNFP-I or a mixture thereof. Preferably the HMO comprises 2′-FL and at least one of LNnT and LNT; at least one of 2′-FL and DFL and at least one of LNnT and LNT (e.g. 2′-FL, DFL and at least one of LNnT and LNT); 2′-FL and 6′-SL; DFL and 6′-SL; 2′-FL, DFL and 6′-SL; 2′-FL, 6′-SL and at least one of LNnT and LNT; and 2′-FL, DFL, 6′-SL and at least one of LNnT and/or LNT.

### DETAILED DESCRIPTION OF THE INVENTION

It has now been surprisingly found that oral or enteral administration of one or more human milk oligosaccharides (HMOs) to a lactose-intolerant, IBS patient prophylactically reduces gastrointestinal IBS symptom severity and/or occurrence in the patient. Further, the administration of the HMOs permits reintroduction of sources of lactose into the diet of the lactose-intolerant, IBS patient. It has also been surprisingly found that the administration of HMOs to a lactose-intolerant, IBS patient reduces the severity and/or occurrence of non-gastrointestinal symptoms in the patient. Therefore, the patient is not only able to better manage IBS symptoms, but may also be able to reintroduce sources of lactose or increase consumption of sources of lactose such as dairy.

The HMOs also preferentially increase the abundance of bifidobacteria in the gastrointestinal tract of the patient, in particular bifidobacteria of the B. adolescentis phylogenetic group, Bifidobacterium longum and/or Bifidobacterium bifidum. The Bifidobacterium longum can be a Bifidobacterium longum longum or a Bifidobacterium longum infantis. These bacteria produce metabolites such as lactate and the short chain fatty acid acetate which in turn can be converted into butyrate by butyrate-producing bacteria. The increase in short chain fatty acids improves digestive health and nourishes the intestinal barrier. A poor intestinal barrier may be a cause of IBS symptoms. Further the increase of bifidobacteria could lead to higher activity of beta-galactosidase, and hence better digestion of the undigested lactose in the colon. In addition, since bifidobacteria are able to inhibit the growth of hydrogen producers such as Escherichia and Clostridium, this shifts the bacterial digestion of lactose away from hydrogen fermentation. This reduces symptoms of lactose intolerance such as excessive gas production.

In this specification, the following terms have the following meanings:

“Bifidobacterium of the B. adolescentis phylogenetic group” means a bacterium selected from a group consisting of Bifidobacterium adolescentis, Bifidobacterium angulatum, Bifidobacterium catenulatum, Bifidobacterium pseudocatenulatum, Bifidobacterium kashiwanohense, Bifidobacterium dentum and Bifidobacterium stercoris (Duranti et al. Appl. Environ. Microbiol. 79, 336 (2013), Bottacini et al. Microbial Cell Fact. 13:S4 (2014)). Preferably, a Bifidobacterium of the B. adolescentis phylogenetic group is Bifidobacterium adolescentis and/or Bifidobacterium pseudocatenulatum.

“Effective amount” means an amount of an HMO sufficient to render a desired outcome in a human. An effective amount can be administered in one or more doses to achieve the desired outcome.

“Enteral administration” means any conventional form for delivery of a composition to a patient that causes the deposition of the composition in the gastrointestinal tract (including the stomach). Methods of enteral administration include feeding through a naso-gastric tube or jejunum tube, oral, sublingual and rectal.

“Human milk oligosaccharide” or “HMO” means a complex carbohydrate found in human breast milk (Urashima et al.: Milk Oligosaccharides. Nova Science Publisher (2011); Chen Adv. Carbohydr. Chem. Biochem. 72, 113 (2015)). The HMOs have a core structure comprising a lactose unit at the reducing end that can be elongated by one or more β-N-acetyl-lactosaminyl and/or one or β-more lacto-N-biosyl units, and which core structure can be substituted by an α L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated

HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose 11 (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II(LNDFH-II), fucosyl-lacto-N-hexaose I (FLNH-I), fucosyl-para-lacto-N-hexaose 1 (FpLNH-I), fucosyl-para-lacto-N-neohexaose II (FpLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-1 I) and disialyl-lacto-N-tetraose (DSLNT).

“Irritable bowel syndrome” (abbreviated as IBS) means a functional bowel disorder in which recurrent abdominal pain is associated with defecation or a change in bowel habits. Disordered bowel habits are typically present (i.e., constipation, diarrhoea or a mix of constipation and diarrhoea), as are symptoms of abdominal bloating/distension. When diagnosed under Rome IV criteria, the patient must experience recurrent abdominal pain on average at least 1 day/week in the last 3 months, and the pain must be associated with two or more of the following criteria: (1) related to defecation; (2) associated with a change in the frequency of stool; and (3) associated with a change in the form (appearance) of stool. Symptom onset should occur at least 6 months prior to diagnosis and symptoms should be present during the last 3 months.

“Lactose intolerance” means the impaired ability to tolerate lactose due to reduced expression or impaired activity of the lactase enzyme in the small intestine. Those affected vary in the amount of lactose they can tolerate before symptoms develop. Common gastrointestinal symptoms include abdominal pain, bloating, diarrhoea, gas, and nausea. Common non-gastrointestinal symptoms include trouble sleeping, headache, lack of energy, fatigue, inability to concentrate, anxiety, depression, and skin rash.

“Microbiota”, “microflora” and “microbiome” mean a community of living microorganisms that typically inhabits a bodily organ or part, particularly the gastrointestinal organs of humans. The most dominant members of the gastrointestinal microbiota include microorganisms of the phyla of Firmicutes, Bacteroidetes, Actinobacteria, Proteobacteria, Synergistetes, Verrucomicrobia, Fusobacteria, and Euryarchaeota; at genus level Bacteroides, Faecalibacterium, Bifidobacterium, Roseburia, Alistipes, Collinsella, Blautia, Coprococcus, Ruminococcus, Eubacterium and Dorea; at species level Bacteroides uniformis, Alistipes putredinis, Parabacteroides merdae, Ruminococcus bromii, Dorea longicatena, Bacteroides caccae, Bacteroides thetaiotaomicron, Eubacterium hallii, Ruminococcus torques, Faecalibacterium prausnitzii, Ruminococcus lactaris, Collinsella aerofaciens, Dorea formicigenerans, Bacteroides vulgatus and Roseburia intestinalis. The gastrointestinal microbiota includes the mucosa-associated microbiota, which is located in or attached to the mucous layer covering the epithelium of the gastrointestinal tract, and luminal-associated microbiota, which is found in the lumen of the gastrointestinal tract.

“Modulating of microbiota” means exerting a modifying or controlling influence on microbiota, for example an influence leading to an increase in the indigenous intestinal abundance of Bifidobacterium, Barnesiella, Faecalibacterium and/or other butyrate producing bacteria. In another example, the influence may lead to a reduction of the intestinal abundance of Ruminococcus gnavus and/or Proteobacteria. “Proteobacteria” are a phylum of Gram-negative bacteria and include a wide variety of pathogenic bacteria, such as Escherichia, Salmonella, Vibrio, Helicobacter, Yersinia and many other notable genera.

“Oral administration” means any conventional form for the delivery of a composition to a human through the mouth. Accordingly, oral administration is a form of enteral administration.

“Preventive treatment” or “prevention” in the present context means treatment given or action taken to diminish the risk of onset or recurrence of a disease.

“Prophylactically reducing symptom severity and/or occurrence” means reducing the severity and/or occurrence of symptoms at a later point in time.

“Relative abundance of a bifidobacteria” means the abundance of a bifidobacteria species relative to other bifidobacteria in the microbiota of the gastrointestinal tract of humans.

“Relative growth of a bifidobacteria” means the growth of a Bifidobacterium species relative to other bifidobacteria in the microbiota in the gastrointestinal tract of humans.

“Secondary prevention” means prevention of onset of the condition in a high-risk patient, or prevention of reoccurrence of symptoms in a patient who has already has the condition. A “high-risk” patient is an individual who is predisposed to developing the condition; for example, a person with a family history of the condition.

“Synthetic composition” means a composition which is artificially prepared and preferably means a composition containing at least one compound that is produced ex vivo chemically and/or biologically, e.g. by means of chemical reaction, enzymatic reaction or recombinantly. In some embodiments, a synthetic composition of the invention may be, but preferably is not, identical with a naturally occurring composition. The synthetic composition typically comprises one or more compounds, including one or more HMOs, that are capable of reducing IBS symptoms in a lactose intolerant, IBS patient. Also, in some embodiments, the synthetic compositions may comprise one or more nutritionally or pharmaceutically active components which do not affect adversely the efficacy of the above-mentioned compounds. Some non-limiting embodiments of a synthetic composition of the invention are also described below.

“Treat” means to address a medical condition or disease with the objective of improving or stabilising an outcome in the person being treated or addressing an underlying nutritional need. Treat therefore includes the dietary or nutritional management of the medical condition or disease by addressing nutritional needs of the person being treated. “Treating” and “treatment” have grammatically corresponding meanings.

“Therapy” means treatment given or action taken to reduce or eliminate symptoms of a disease or pathological condition.

The HMOs can be isolated or enriched by well-known processes from milk(s) secreted by mammals including, but not limited to human, bovine, ovine, porcine, or caprine species. The HMOs can also be produced by well-known processes using microbial fermentation, enzymatic processes, chemical synthesis, or combinations of these technologies. As examples, using chemistry LNnT can be made as described in WO 2011/100980 and WO 2013/044928, LNT can be synthesized as described in WO 201 2/1 5591 6 and WO 2013/044928, a mixture of LNT and LNnT can be made as described in WO 2013/091660, 2′-FL can be made as described in WO 2010/115934 and WO 2010/115935, 3-FL can be made as described in WO 2013/139344, 6′-SL and salts thereof can be made as described in WO 2010/100979, sialylated oligosaccharides can be made as described in WO 2012/113404 and mixtures of human milk oligosaccharides can be made as described in WO 2012/113405. As examples of enzymatic production, sialylated oligosaccharides can be made as described in WO 2012/007588, fucosylated oligosaccharides can be made as described in WO 2012/127410, and advantageously diversified blends of human milk oligosaccharides can be made as described in WO 2012/156897 and WO 2012/156898. Biotechnological methods which describe how to make core (non-fucosylated neutral) human milk oligosaccharides optionally substituted by fucose or sialic acid using genetically modified E. coli con be found in WO 01/04341 and WO 2007/101862.

The HMO may be a single HMO or a mixture of any HMOs suitable for the purpose of the invention.

In one embodiment, the mixture comprises, consists of or essentially consists of, neutral HMOs, preferably at least a first neutral HMO and at least a second neutral HMO, wherein the first neutral HMO is a fucosylated neutral HMO and the second neutral HMO is a core HMO (also referred to as non-fucosylated neutral HMO). Particularly, the mixture of HMOs may contain a fucosylated HMO selected from the list consisting of 2′-FL, 3-FL, DFL, LNFP-I, LNFP-II, LNFP-III, LNFP-V, LNDFH-I, LNDFH-II, LNDFH-III, FLNH-I, FLNH-II, FLNnH, FpLNH-I and F-pLNnH II, and a core HMO selected from the list consisting of LNT, LNnT, LNH, LNnH, pLNH and pLNnH. More preferably, the mixture of neutral HMOs contains, consists of or essentially consists of, a fucosylated HMO selected from the list consisting of 2′-FL, 3-FL and DFL, and a core HMO selected from the list consisting of LNT and LNnT; advantageously the mixture comprises, consists of or essentially consists of, 2′-FL and at least one of LNnT and LNT; or at least one of 2′-FL and DFL and at least one of LNnT and LNT; or 2′-FL, DFL and at least one of LNnT and LNT.

In other embodiment, the mixture comprises, consists of or essentially consists of, at least a first (acidic) HMO and at least a second (neutral) HMO, wherein the first (acidic) HMO is selected from the list consisting of 3′-SL, 6′-SL and FSL and the second (neutral) HMO is selected from the list consisting of 2′-FL, 3-FL, DFL, LNT and LNnT; advantageously the mixture comprises, consists of or essentially consists of, 2′-FL and 6′-SL; or 6′-SL and at least one of 2′-FL and DFL; or 2′-FL, 6′-SL and at least one of LNnT and LNT; or 2′-FL, DFL, 6′-SL and at least one of LNnT and/or LNT.

In one embodiment, the synthetic composition can be in the form of a nutritional composition. For example, the nutritional composition can be a food composition, a rehydration solution, a medical food or food for special medical purposes, a nutritional supplement and the like. The nutritional composition can contain sources of protein, lipids and/or digestible carbohydrates and can be in powdered or liquid forms. The composition can be designed to be the sole source of nutrition or as a nutritional supplement.

Suitable protein sources include milk proteins, soy protein, rice protein, pea protein and oat protein, or mixtures thereof. Milk proteins can be in the form of milk protein concentrates, milk protein isolates, whey protein or casein, or mixtures of both. The protein can be whole protein or hydrolysed protein, either partially hydrolysed or extensively hydrolysed. Hydrolysed protein offers the advantage of easier digestion which can be important for humans with inflamed or compromised GI tracts. The protein can also be provided in the form of free amino acids. The protein can comprise about 5% to about 30% of the energy of the nutritional composition, normally about 10% to 20%. Ideally, the source of protein does not include excessive amounts of lactose.

The protein source can be a source of glutamine, threonine, cysteine, serine, proline, or a combination of these amino acids. The glutamine source can be a glutamine dipeptide and/or a glutamine enriched protein. Glutamine can be included due to the use of glutamine by enterocytes as an energy source. Threonine, serine and proline are important amino acids for the production of mucin. Mucin coats the GI tract and can improve intestinal barrier function and mucosal healing. Cysteine is a major precursor of glutathione, which is key for the antioxidant defences of the body.

Suitable digestible carbohydrates include maltodextrin, hydrolysed or modified starch or corn starch, glucose polymers, corn syrup, corn syrup solids, high fructose corn syrup, rice-derived carbohydrates, pea-derived carbohydrates, potato-derived carbohydrates, tapioca, sucrose, glucose, fructose, sucrose, honey, sugar alcohols (e.g. maltitol, erythritol, sorbitol), or mixtures thereof. Preferably the composition is reduced in or free from added lactose or other FODMAP carbohydrates. Generally digestible carbohydrates provide about 35% to about 55% of the energy of the nutritional composition. A suitable digestible carbohydrate is a low dextrose equivalent (DE) maltodextrin.

Suitable lipids include medium chain triglycerides (MCT) and long chain triglycerides (LCT). Preferably the lipid is a mixture of MCTs and LCTs. For example, MCTs can comprise about 30% to about 70% by weight of the lipids, more specifically about 50% to about 60% by weight. MCTs offer the advantage of easier digestion which can be important for humans with inflamed or compromised GI tracts. Generally, the lipids provide about 35% to about 50% of the energy of the nutritional composition. The lipids can contain essential fatty acids (omega-3 and omega-6 fatty acids). Preferably these polyunsaturated fatty acids provide less than about 30% of total energy of the lipid source.

Suitable sources of long chain triglycerides are rapeseed oil, sunflower seed oil, palm oil, soy oil, milk fat, corn oil, high oleic oils, and soy lecithin. Fractionated coconut oils are a suitable source of medium chain triglycerides. The lipid profile of the nutritional composition is preferably designed to have a polyunsaturated fatty acid omega-6 (n-6) to omega-3 (n-3) ratio of about 4:1 to about 10:1. For example, the n-6 to n-3 fatty acid ratio can be about 6:1 to about 9:1.

The nutritional composition may also include vitamins and minerals. If the nutritional composition is intended to be a sole source of nutrition, it preferably includes a complete vitamin and mineral profile. Examples of vitamins include vitamins A, B-complex (such as B1, B2, B6 and B12), C, D, E and K, niacin and acid vitamins such as pantothenic acid, folic acid and biotin. Examples of minerals include calcium, iron, zinc, magnesium, iodine, copper, phosphorus, manganese, potassium, chromium, molybdenum, selenium, nickel, tin, silicon, vanadium and boron.

The nutritional composition can also include a carotenoid such as lutein, lycopene, zeaxanthin, and beta-carotene. The total amount of carotenoid included can vary from about 0.001 μg/ml to about 10 μg/ml. Lutein can be included in an amount of from about 0.001 μg/ml to about 10 μg/ml, preferably from about 0.044 μg/ml to about 5 μg/ml of lutein. Lycopene can be included in an amount from about 0.001 μg/ml to about 10 μg/ml, preferably about 0.0185 μg/ml to about 5 μg/ml of lycopene. Beta-carotene can comprise from about 0.001 μg/ml to about 10 mg/ml, for example about 0.034 μg/ml to about 5 μg/ml of beta-carotene.

The nutritional composition preferably also contains reduced concentrations of sodium; for example, from about 300 mg/I to about 400 mg/I. The remaining electrolytes can be present in concentrations set to meet needs without providing an undue renal solute burden on kidney function. For example, potassium is preferably present in a range of about 1180 to about 1300 mg/I; and chloride is preferably present in a range of about 680 to about 800 mg/I.

The nutritional composition can also contain various other conventional ingredients such as preservatives, emulsifying agents, thickening agents, buffers, fibres and prebiotics (e.g. fructooligosaccharides, galactooligosaccharides), probiotics (e.g. B. animalis subsp. lactis BB-12, B. lactis HNO19, B. lactis Bi07, B. infantis ATCC 15697, L. rhamnosus GG, L. rhamnosus HNOOI, L. acidophilus LA-5, L. acidophilus NCFM, L. fermentum CECT5716, B. longum BB536, B. longum AH1205, B. longum AH1206, B. breve M-16V, L. reuteri ATCC 55730, L. reuteri ATCC PTA-6485, L. reuteri DSM 17938), antioxidant/anti-inflammatory compounds including tocopherols, carotenoids, ascorbate/vitamin C, ascorbyl palmitate, polyphenols, glutathione, and superoxide dismutase (melon), other bioactive factors (e.g. growth hormones, cytokines, TFG-β), colorants, flavours, and stabilisers, lubricants, and so forth.

The nutritional composition can be formulated as a soluble powder, a liquid concentrate, or a ready-to-use formulation. The composition can be fed to a human in need via a nasogastric tube or orally. Various flavours and other additives can also be present.

The nutritional compositions can be prepared by any commonly used manufacturing techniques for preparing nutritional compositions in solid or liquid form. For example, the composition can be prepared by combining various feed solutions. A protein-in-fat feed solution can be prepared by heating and mixing the lipid source and then adding an emulsifier (e.g. lecithin), fat soluble vitamins, and at least a portion of the protein source while heating and stirring. A carbohydrate feed solution is then prepared by adding minerals, trace and ultra-trace minerals, thickening or suspending agents to water while heating and stirring. The resulting solution is held for 10 minutes with continued heat and agitation before adding carbohydrates (e.g. the HMOs and digestible carbohydrate sources). The resulting feed solutions are then blended together while heating and agitating and the pH adjusted to 6.6-7.0, after which the composition is subjected to high-temperature short-time processing during which the composition is heat treated, emulsified and homogenized, and then allowed to cool. Water soluble vitamins and ascorbic acid are added, the pH is adjusted to the desired range if necessary, flavours are added, and water is added to achieve the desired total solid level.

For a liquid product, the resulting solution can then be aseptically packed to form an aseptically packaged nutritional composition. In this form, the nutritional composition can be in ready-to-feed or concentrated liquid form. Alternatively, the composition can be spray-dried and processed and packaged as a reconstitutable powder.

When the nutritional product is a ready-to-feed nutritional liquid, it may be preferred that the total concentration of HMOs in the liquid, by weight of the liquid, is from about 0.1% to about 1.5%, including from about 0.2% to about 1.0%, for example from about 0.3% to about 0.7%. When the nutritional product is a concentrated nutritional liquid, it may be preferred that the total concentration of HMOs in the liquid, by weight of the liquid, is from about 0.2% to about 3.0%, including from about 0.4% to about 2.0%, for example from about 0.6% to about 1.5%.

In another embodiment, the nutritional composition is in a unit dosage form. The unit dosage form can contain an acceptable food-grade carrier, e.g. phosphate buffered saline solution, mixtures of ethanol in water, water and emulsions such as an oil/water or water/oil emulsion, as well as various wetting agents or excipients. The unit dosage form can also contain other materials that do not produce an adverse, allergic or otherwise unwanted reaction when administered to a human. The carriers and other materials can include solvents, dispersants, coatings, absorption promoting agents, controlled release agents, and one or more inert excipients, such as starches, polyols, granulating agents, microcrystalline cellulose, diluents, lubricants, binders, and disintegrating agents. Preferably the unit dosage form comprises primarily HMOs with a minimum amount of binders and/or excipients. Unit dosage forms are particularly suitable when nutritionally incomplete or not intended as a sole source of nutrition.

A unit dosage form can be administered orally, e.g. as a tablet, capsule, or pellet containing a predetermined amount of the mixture, or as a powder or granules containing a predetermined concentration of the mixture or a gel, paste, solution, suspension, emulsion, syrup, bolus, electuary, or slurry, in an aqueous or non-aqueous liquid, containing a predetermined concentration of the mixture. An orally administered composition can include one or more binders, lubricants, inert diluents, flavouring agents, and humectants. An orally administered composition such as a tablet can optionally be coated and can be formulated to provide sustained, delayed or controlled release of the HMO.

A unit dosage form can also be administered by naso-gastric tube or direct infusion into the GI tract or stomach.

A unit dosage form can also include therapeutic agents such as antibiotics, probiotics, analgesics, and anti-inflammatory agents.

The proper dosage of a nutritional composition for a human can be determined in a conventional manner, based upon factors such as the concentration of HMO, the human's condition, immune status, body weight and age. In some cases, the dosage will be such that the HMO is delivered at a concentration similar to that found in human breast milk. The required amount of HMO would generally be in the range from about 1 g to about 15 g per day, in certain embodiments from about 2 g to about 10 g per day, for example about 3 g to about 7 g per day. Appropriate dose regimes can be determined by methods known to those skilled in the art.

In further embodiment, the HMO can be formulated as a pharmaceutical composition. The pharmaceutical composition can contain a pharmaceutically acceptable carrier, e.g. phosphate buffered saline solution, mixtures of ethanol in water, water and emulsions such as an oil/water or water/oil emulsion, as well as various wetting agents or excipients. The pharmaceutical composition can also contain other materials that do not produce an adverse, allergic or otherwise unwanted reaction when administered to a human. The carriers and other materials can include solvents, dispersants, coatings, absorption promoting agents, controlled release agents, and one or more inert excipients, such as starches, polyols, granulating agents, microcrystalline cellulose, diluents, lubricants, binders, and disintegrating agents.

The pharmaceutical compositions can be administered orally, e.g. as a tablet, capsule, or pellet containing a predetermined amount, or as a powder or granules containing a predetermined concentration or a gel, paste, solution, suspension, emulsion, syrup, bolus, electuary, or slurry, in an aqueous or non-aqueous liquid, containing a predetermined concentration. Orally administered compositions can include binders, lubricants, inert diluents, flavouring agents, and humectants. Orally administered compositions such as tablets can optionally be coated and can be formulated to provide sustained, delayed or controlled release of the mixture therein.

The pharmaceutical compositions can also be administered by rectal suppository, aerosol tube, naso-gastric tube or direct infusion into the GI tract or stomach.

The pharmaceutical compositions can also include therapeutic agents such as antibiotics, probiotics, analgesics, and anti-inflammatory agents. The proper dosage of a pharmaceutical composition can be determined in a conventional manner, based upon factors such the concentration of the HMO, the patient's condition, immune status, body weight and age. In some cases, the dosage will be such that the HMO is delivered at a concentration similar to that found in human breast milk. The required amount of HMO would generally be in the range from about 1 g to about 15 g per day, in certain embodiments from about 2 g to about 10 g per day, for example about 3 g to about 7 g per day. Appropriate dose regimes can be determined by methods known to those skilled in the art.

For prophylactically reducing gastrointestinal IBS symptom severity and/or occurrence in a lactose-intolerant, IBS patient, reintroducing sources of lactose into the diet of a lactose-intolerant, IBS patient, and/or reducing the severity and/or occurrence of non-gastrointestinal symptoms in a lactose-intolerant, IBS patient, the amount of HMO(s) required to be administered will vary depending upon factors such as the risk and severity of the IBS symptoms, any underlying medical condition or disease, age, the form of the composition, and other medications being administered. Further the amount may vary depending upon whether the HMOs are being used prophylactically (when the dose may be higher) or whether the HMOs are being used in during maintenance (when the dose may be lower). However, the required amount can be readily set by a medical practitioner and would generally be in the range from about 1 g to about 15 g per day, in certain embodiments from about 2 g to about 10 g per day, for example from about 3 g to about 7 g per day. An appropriate dose can be determined based on several factors, including, for example, body weight and/or condition, the severity of the IBS symptoms being managed, other ailments and/or diseases, the incidence and/or severity of side effects and the manner of administration. Appropriate dose ranges may be determined by methods known to those skilled in the art. During an initial phase, the dosing can be higher (for example 3 g to 15 g per day, preferably 3 mg to 10 g per day). During a maintenance phase, the dosing can be reduced (for example, 1 g to 10 g per day, preferably 2 g to 7.5 g per day).

### EXAMPLES

### Example 1

A total of 317 male and female patients are recruited to participate in the study from 17 clinics. Patients are eligible to participate in the study if they are over 18 years of age at screening; have a current clinical diagnosis of IBS from a healthcare provider; and meet Rome IV IBS diagnostic criteria. Patients are excluded from participation if they are diagnosed with coeliac disease, diverticulitis, inflammatory bowel disease or Clostridium difficile infection by a medical doctor.

All patients attend a screening visit at the clinic where written informed consent is obtained, and inclusion and exclusion criteria are checked. After inclusion in the trial, the patients fill out an electronic baseline survey (survey 1). The survey includes a number of validated questionnaires such as the Bristol Stool Form Scale (BSFS), IBS Symptom Severity Score (IBS-SSS), IBS specific Gastrointestinal symptom Rating Scale (GSRS-IBS), and IBS Quality of Life (IBS-QoL). Patients also indicate whether they are lactose intolerant. Of the 317 patients recruited, 67 indicate lactose intolerance.

Upon completion of survey 1, the patients consume a HMO product made up of a combination of two HMOs (2′-FL and LNnT in a 4:1 mix) each day for a period of 4 weeks. The HMO product is provided in single-serve stick packs each containing 5 g of the two HMOs with each patient consuming the contents of 1 stick pack per day.

After 4 weeks, the patients complete a second electronic survey (survey 2) which includes the same validated questionnaires and questions on compliance to intervention, perception of the HMO product, and adverse events (AEs). The patients then consume the HMO product on a daily basis for the next 4 weeks. After the 4 weeks, the patients complete a third electronic survey (survey 3) which includes the same validated questionnaires and questions on compliance to intervention, perception of the HMO product, and adverse events (AEs). The patients again consume the HMO products for 4 weeks followed by a final survey (survey 4). Survey 4 includes the same validated questionnaires and questions on compliance to intervention, perception of the HMO product, and adverse events (AEs).

For both primary and secondary outcomes, a two-factored, mixed-design Analysis of Variance (ANOVA) is used for analysis, with the three IBS subtypes as the between-subjects factor and two timepoints (baseline vs week 12) as within-subject factor. The analysis is performed using an intent-to-treat (ITT) methodology, including data from all patients who received a shipment of the product regardless of whether they started intervention, and carrying last observation obtained forward to fill any missing data points from discontinued patients.

The primary efficacy endpoint in the trial is the mean change from baseline in the proportion of bowel movements with abnormal stool consistency as measured by BSFS. Secondary endpoints include change from baseline in overall IBS severity measured by IBS-SSS, severity of gastrointestinal symptoms measured by GSRS-IBS, and patients' health related quality of life measured by IBS-QoL. Safety is assessed by collecting and monitoring adverse events throughout the course of the trial.

A total of 49 lactose intolerant patients complete the trial. The lactose intolerant patients show a significant reduction from baseline to 12 weeks in total percentage of abnormal bowel movements (Bristol Stool Form Scale types 1, 2, 6, or 7), and overall IBS symptom severity. The patients also show a significant improvement in health-related quality of life. Most of the symptom improvement occurs in the first 4 weeks of intervention. The improvement in symptoms in the lactose intolerant subgroup is similar to other IBS patients. The results are shown in the following table:

Percentage of abnormal bowel movements in the past 4 weeks measured by BSFS

| | Overall (n = 317) | | Lactose intolerant (n = 67) | | Lactose tolerant (n = 250) | |
|---|---|---|---|---|---|---|
| Baseline | 90.7 | [88.5-92.9] | 91.3 | [85.2-97.5] | 90.5 | [88.3-92.8] |
| Week 4 | 60.5* | [57.5-63.6] | 65.1* | [58.2-72] | 59.3* | [55.9-62.7] |
| Week 8 | 57.4* | [54.2-60.5] | 56.7* | [48.4-65.1] | 57.5* | [54.2-60.9] |
| Week 12 | 57.3* | [54.0-60.6] | 55.7* | [47.7-63.6] | 57.7* | [54.2-61.3] |

Total IBS Symptom Severity Score

| | | | | | | |
|---|---|---|---|---|---|---|
| Baseline | 323 | [314-332] | 309 | [290-329] | 327 | [317-337] |
| Week 4 | 178* | [167-188] | 174* | [148-199] | 179* | [168-191] |
| Week 8 | 151* | [140-161] | 148* | [121-175] | 151* | [140-163] |
| Week 12 | 144* | [133-155] | 143* | [115-171] | 145* | [133-156] |

Total IBS Quality of Life Score

| | | | | | | |
|---|---|---|---|---|---|---|
| Baseline | 50.4 | [48.0-52.8] | 49.4 | [43.5-55.3] | 50.6 | [48.0-53.3] |
| Week 4 | 70.1* | [67.9-72.2] | 65.7* | [60.1-71.2] | 71.3* | [69.0-73.6] |
| Week 8 | 74.3* | [72.0-76.6] | 67.5* | [61.5-73.6] | 76.1* | [73.7-78.5] |
| Week 12 | 74.6* | [72.3-76.9] | 70.7* | [64.7-76.6] | 75.7* | [73.2-78.1] |

*P < 0.0001 compared to baseline values.
The 67 lactose intolerant patients report adverse events similar to the lactose tolerant patients. Most are assessed to be mild.

### Example 2

The ability of different HMOs to increase the abundance of bifidobacteria in IBS faeces is assessed in a batch culture fermentation system. The following HMOs are tested: 2′-FL, LNnT, LNT, 3′-SL, 6′-SL and a mix of 2′-FL and DFL.

Faecal samples are collected from IBS-D patients, IBS-C patients and healthy volunteers. Batch fermenters are set up (300 ml working volume) and each fermenter is aseptically inoculated with a faecal sample, a basal culture medium and one of the HMOs. Temperature is kept constant at 37° C. (human body temperature) by means of a circulating water bath and the pH is regulated to 6.8.

The batch fermenters are run for 48 hours with samples taken at 0 hour, 8 hours, and 48 hours. The samples are subjected to analysis using:
Fluorescent in situ hybridisation (FISH) combined with flow cytometry (Flow-FISH) to enumerate the bacterial population. Eleven genotypic probes are used targeting specific regions of 16S rRNA of predominant bacterial taxa of the gut microbiota including total bacteria.

The abundance of bifidobacteria increases in the IBS and healthy faecal samples with the addition of HMOs. Bifidobacteria is able to consume lactose without producing gases leading to lactose intolerant symptoms indicating that the HMOs are suitable for increasing bifidobacteria in lactose intolerant IBS patients.

### Example 3

The HMOs 2′-FL and LNnT are introduced into a rotary blender in a 4:1 mass ratio. An amount of 0.25 w % of magnesium stearate is introduced into the blender and the mixture blended for 10 minutes. The mixture is then agglomerated in a fluidised bed and filled into 5 gram stick packs and the packs are sealed.

### Example 4

The procedure of example 3 is repeated except that the agglomeration step is omitted.

1-22. (canceled)

23. A method comprising:
selecting a non-infant human with irritable bowel syndrome and lactose intolerance, the non-infant human following a diet that avoids one or more selected lactose sources that have previously resulted in the non-infant human experiencing a reference level of one or more symptoms of the lactose intolerance;
selecting an effective amount of one or more human milk oligosaccharides (HMOs) chosen from the group consisting of 2′-fucosyllactose (2′-FL), difucosyllactose (DFL), 3-fucosyllactose (3-FL), lacto-N-fucopentaose I (LNFP-I), lacto-N-tetraose (LNT), lacto-N-neotetraose (LNnT), 3′-sialyllactose (3′-SL), 6′-sialyllactose (6′-SL), and combinations thereof, the chosen one or more HMOs effective for modulating the gut microbiota of the non-infant human;
administering the effective amount of the chosen HMOs to the non-infant human during an initial phase;
reintroducing one or more of the selected lactose sources into the diet of the non-infant human during the initial phase; and
reducing frequency and/or severity of the one or more symptoms experienced by the non-infant human as a result of reintroducing the at least one of the selected lactose sources into the diet of the non-infant human relative to the reference level.

24. The method of claim 23, wherein the selected lactose sources reintroduced during the initial phase in which the chosen one or more HMOs are administered comprise lactose-containing dairy foods selected from milks, soft cheeses, and ice creams.

25. The method of claim 23, wherein the chosen one or more HMOs comprise:
at least one fucosylated neutral HMO selected from 2′-FL and DFL; and
at least one non-fucosylated neutral HMO selected from LNnT and LNT.

26. The method of claim 23, wherein a total daily initial phase dosage administered of the chosen one or more HMOs is from about 3 g to about 10 g per day.

27. The method of claim 26, wherein during a maintenance phase following the initial phase, a total daily maintenance phase dosage administered of the chosen one or more HMOs is from about 2 g to about 5 g per day.

28. The method of claim 23, wherein the one or more of the selected lactose sources are reintroduced into the diet of the non-infant human after administering the chosen HMOs for a period of at least one week.

29. The method of claim 23, wherein the one or more of the selected lactose sources are reintroduced into the diet of the non-infant human before administering the chosen HMOs during the initial phase.

30. The method of claim 23, wherein modulating the gut microbiota of the non-infant human comprises increasing the relative abundance of Bifidobacterium adolescentis in the colon of the non-infant human.

31. The method of claim 23, further comprising increasing levels of butyrate in the colon of the non-infant human in response to administering the effective amount of the chosen HMOs.

32. The method of claim 23, further comprising enhancing lactose fermentation by promoting beta-galactosidase activity in the colon of the non-infant human in response to administering the effective amount of the chosen HMOs.

33. The method of claim 23, wherein reducing frequency and/or severity of the one or more symptoms of the lactose intolerance comprises reducing frequency and/or severity of non-gastrointestinal symptoms selected from depression, fatigue, lack of energy, and combinations thereof.

34. The method of claim 23, wherein reducing frequency and/or severity of the one or more symptoms of the lactose intolerance comprises reducing frequency and/or severity of non-gastrointestinal symptoms selected from headache, anxiety, trouble sleeping, and combinations thereof.

35. The method of claim 23, wherein reducing frequency and/or severity of the one or more symptoms of the lactose intolerance comprises reducing frequency and/or severity of skin rash.

36. A method comprising:
selecting a non-infant human with irritable bowel syndrome experiencing one or more symptoms of lactose intolerance;
selecting an effective amount of one or more human milk oligosaccharides (HMOs) chosen from the group consisting of 2′-fucosyllactose (2′-FL), difucosyllactose (DFL), 3-fucosyllactose (3-FL), lacto-N-fucopentaose I (LNFP-I), lacto-N-tetraose (LNT), lacto-N-neotetraose (LNnT), 3′-sialyllactose (3′-SL), 6′-sialyllactose (6′-SL), and combinations thereof, the chosen one or more HMOs effective for modulating the gut microbiota of the non-infant human; and
reducing frequency and/or severity of the one or more symptoms associated with the lactose intolerance by administering the effective amount of the chosen HMOs to the non-infant human during an initial phase.

37. The method of claim 36, wherein the chosen one or more HMOs comprise:
at least one fucosylated neutral HMO selected from 2′-FL and DFL; and
at least one non-fucosylated neutral HMO selected from LNnT and LNT.

38. The method of claim 36, wherein a total daily initial phase dosage administered of the chosen one or more HMOs is from about 3 g to about 10 g per day.

39. The method of claim 36, wherein modulating the gut microbiota of the non-infant human comprises increasing the relative abundance of Bifidobacterium adolescentis and butyrate producing bacteria in the colon of the non-infant human in response to administering the effective amount of the chosen HMOs.

40. The method of claim 36, further comprising enhancing lactose fermentation by promoting beta-galactosidase activity in the colon of the non-infant human in response to administering the effective amount of the chosen HMOs.

41. The method of claim 36, wherein reducing frequency and/or severity of the one or more symptoms of the lactose intolerance comprises reducing frequency and/or severity of non-gastrointestinal symptoms selected from depression, fatigue, lack of energy, headache, anxiety, trouble sleeping and combinations thereof.

42. The method of claim 36, wherein reducing frequency and/or severity of the one or more symptoms of the lactose intolerance comprises reducing frequency and/or severity of skin rash.
